(19) United States
(12) Patent Application Publication
(10) Pub. No.: US 2022/0142877 A1
(43) Pub. Date: May 12, 2022

(54) COSMETIC OR PERSONAL CARE FORMULATIONS CONTAINING POROUS METAL OXIDE SPHERES

(71) Applicant: BASF COLORS & EFFECTS GMBH, Ludwigshafen am Rhein (DE)

(72) Inventors: James Paul NEWHOUSE, Tarrytown, NY (US); Charmain E. HOFFSTEAD-FORDYCE, Tarrytown, NY (US); Rupa Hiremath DARJI, Tarrytown, NY (US); Michael David BURKE, Tarrytown, NY (US); Keith TASK, Beachwood, OH (US)

(21) Appl. No.: 17/435,879

(22) PCT Filed: Mar. 12, 2020
(86) PCT No.: PCT/EP2020/056681

§ 371 Date: Sep. 2, 2021

Related U.S. Application Data
(60) Provisional application No. 62/817,193, filed on Mar. 12, 2019.

(51) Int. Cl. A61K 8/02; A61Q 17/04; A61Q 1/10; A61Q 1/06; A61Q 1/12; A61Q 5/02; A61Q 3/02; A61Q 1/04; A61Q 19/10; A61K 8/29

(52) U.S. Cl. CPC A61K 8/0279; A61K 8/025; A61Q 17/04; A61Q 1/10; A61Q 1/06; A61Q 1/12; A61K 2800/413; A61Q 3/02; A61Q 1/04; A61Q 19/10; A61K 8/29; A61K 2800/651; A61Q 5/02

(57) ABSTRACT

A cosmetic or personal care formulation includes a carrier and porous metal oxide spheres wherein, the porous metal oxide spheres have, e.g., an average diameter of about 0.5 μm to about 100 μm and an average porosity of about 0.10 to about 0.80; the porous metal oxide spheres have one or more population of pores each having an average pore diameter, wherein each population has a different average pore diameter and wherein the average pore diameters are, e.g., from about 50 nm to about 999 nm.

### FIELD

The present technology is generally related to cosmetic or personal care formulations that contain structural colorans. More specifically, the technology is related to cosmetic or personal care formulations that contain porous metal oxide spheres (e.g., microspheres), methods of their preparation and uses thereof.

### SUMMARY

In one aspect, a cosmetic or personal care formulation includes a carrier and porous metal oxide spheres (e.g., microspheres) wherein, the porous metal oxide spheres have, e.g., an average diameter of about 0.5 μm to about 100 μm and an average porosity of about 0.10 to about 0.80; the porous metal oxide spheres having one or more population(s) of pores each having an average pore diameter, wherein each population has a different average pore diameter and wherein the average pore diameters are, e.g., from about 50 nm to about 999 nm. Illustrative cosmetic or personal care formulations include, but are not limited to, a lipstick, lip crème, lip liner, lip gloss, eyeshadow, foundation, eyeliner, nail enamel, nail polish, concealer, cream, and other cosmetics formulations.

In any of the embodiments herein, the porous metal oxide spheres (e.g., microspheres) may impart an effect to the cosmetic or personal care formulation such that the observed color is angle dependent, angle independent, stimuli-responsive. In any of the embodiments herein, the porous metal oxide spheres may impart strong light scattering or diffusion behavior to the formulations, or the formulation may appear to changes from a transparent to opaque appearance, or depending on viewing angle the perceived color may change from one quadrant to another in Lab color space. Where the effect is one of stimuli-responsiveness, the stimuli may include, but is not limited to, changes in pH, pressure, ultraviolet light, visible, near-infra red light, temperature, and/or electric current.

### DETAILED DESCRIPTION

Various embodiments are described hereinafter. It should be noted that the specific embodiments are not intended as an exhaustive description or as a limitation to the broader aspects discussed herein. One aspect described in conjunction with a particular embodiment is not necessarily limited to that embodiment and can be practiced with any other embodiment(s).

As used herein, “about” will be understood by persons of ordinary skill in the art and will vary to some extent depending upon the context in which it is used. If there are uses of the term which are not clear to persons of ordinary skill in the art, given the context in which it is used, “about” will mean up to plus or minus 10% of the particular term.

The use of the terms “a” and “an” and “the” and similar referents in the context of describing the elements (especially in the context of the following claims) are to be construed to cover both the singular and the plural, unless otherwise indicated herein or clearly contradicted by context. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the embodiments and does not pose a limitation on the scope of the claims unless otherwise stated. No language in the specification should be construed as indicating any non-claimed element as essential.

As used herein, particle size is synonymous with particle diameter and is determined for instance by scanning electron microscopy (SEM) or transmission electron microscopy (TEM) as described in “Quantitative Analysis of Micron-Scale and Nano-Scale Pore Throat Characteristics of Tight Sandstone Using Matlab”, B. Jiu et al., Applied Sciences, 2018, 8, 1272. In addition, pore diameter is determined for instance by SEM or TEM as described in “Porosity and its measurment”, L. Espinal, Characterization of Materials, edited by Elton N. Kaufmann, 2012. Average particle size is synonymous with D50, meaning half of the population resides above this point, and half below. Particle size refers to primary particles. Particle size may be measured by laser light scattering techniques, with dispersions or dry powders.

Mercury porosimetry analysis may be used to characterize the porosity of the microspheres. Mercury porosimetry applies controlled pressure to a sample immersed in mercury. External pressure is applied for the mercury to penetrate into the voids/pores of the material. The amount of pressure required to intrude into the voids/pores is inversely proportional to the size of the voids/pores. The mercury porosimeter generates volume and pore size distributions from the pressure versus intrusion data generated by the instrument using the Washburn equation. For example, porous silica microspheres containing voids/pores with an average size of 165 nm have an average porosity of 0.8.

The term “bulk sample” means a population of spheres (e.g., microspheres). For example, a bulk sample of spheres is simply a bulk population of microspheres, for instance ≥0.5 mg, ≥0.7 mg, ≥1.0 mg, ≥2.5 mg, ≥5.0 mg, ≥10.0 mg or ≥25.0 mg. A bulk sample of microspheres may be substantially free of other components. The term “porous microspheres” may mean a bulk sample.

The phrase “exhibits color observable by the human eye” means color will be observed by an average person. This may be for any bulk sample distributed over any surface area, for instance a bulk sample distributed over a surface area of from any of about 1 cm², about 2 cm², about 3 cm², about 4 cm², about 5 cm² or about 6 cm² to any of about 7 cm², about 8 cm², about 9 cm², about 10 cm², about 11 cm², about 12 cm², about 13 cm², about 14 cm² or about 15 cm². It may also mean observable by a CIE 1931 2° standard observer and/or by a CIE 1964 10° standard observer. The background for color observation may be any background, for instance a white background, black background or a dark background anywhere between white and black.

The term “of” may mean “comprising”, for instance “a liquid dispersion of” may be interpreted as “a liquid dispersion comprising”.

The terms “microspheres”, “nanospheres”, “droplets”, etc., referred to herein may mean for example a plurality thereof, a collection thereof, a population thereof, a sample thereof or a bulk sample thereof.

The term “micro” or “micro-scaled” means from about 0.5 μm to about 999 μm. The term “nano” or “nano-scaled” means from about 1 nm to about 999 nm.

The terms “spheres” and “particles” may be interchangeable.

The term “monodisperse” in reference to a population of spheres (e.g., microspheres or nanospheres) means particles having generally uniform shapes and generally uniform diameters. A present monodisperse population of spheres for instance may have 90%, 91%, 92%, 93%, 94%, 95%, 96%, 97%, 98% or 99% of the particles by number having diameters within ±7%, ±6%, ±5%, ±4%, ±3%, ±2% or ±1% of the average diameter of the population. The term “monodisperse polymer particles” refers to a population of monodisperse polymer particles.

The term “polydisperse” in reference to pheres means a sample comprising a first monodisperse population having a first average diameter and at least a second monodisperse population having a second average diameter, the first and second diameters being different. A polydisperse sample of spheres contains at least two monodisperse populations and may contain 3, 4, 5, 6, etc. monodisperse populations, each having a different average particle size. A polydisperse sample having only first and second monodisperse polymer nanospheres is a “bimodal” sample, having a bimodal particle size distribution.

The term “substantially free of other components” means for example containing ≤5%, ≤4%, ≤3%, ≤2%, ≤1% or ≤0.5% by weight of other components. Likewise, the term “substantially no” means little or no.

Provided herein are cosmetic and personal care formulations that include structural colorants that are based upon metal oxide spheres (e.g., microspheres). The spheres have porous surfaces containing uniform pore diameters. The size and structure of the uniform pores on the surface of the spheres impart various colorant properties to the materials. For example, the properties imparted to the cosmetics and personal care formulations include angle-dependent colors which may provide extended color travel; angle-independent colors that provide the same color across all viewing angles (this effect is not typical of color effect pigments); strong scattering and diffusion properties which may provide high white coloration without the use of traditional pigments such as titanium dioxide; colors that change depending on the specific ingredients in the formulations that accompany the structural colorants which gives formulators the ability to produce a variety of shades of a product using only a single structural colorant (i.e. pigment); the ability to transition the color from transparent to visible or opaque which may provide the ability to have a cosmetic or personal care product that is colorless or clear in when in a storage container, but for which color is observed when put on the skin; four quadrant color (i.e. the color change may travel through all four quadrants of a-b color space) which may provide a true holographic effect without the use of diffraction grating materials; color pigments with added functionality such as UV indicators, actives delivery, or textural properties, which may provide for the protection of, or controlled release of, active materials to the skin, and where the structural colorants may impart a silky texture for application while also providing the coloration to the cosmetic; and color-responsive pigments which can change color in response to an external stimuli such as pH, pressure, ultraviolet light exposure, visible, light exposure, near-infra red light exposure, temperature, or electricity to achieve a wide array of colors depending on the external stimuli the cosmetic encounters.

The cosmetics and personal care formulations described herein include, but are not limited to, sunscreens, body creams, body lotions, body sprays, nail enamels, lip colors, eye makeup colors, hair colors, face colors, cleansers, and pressed powders. The form of the cosmetics and personal care formulations may include a cream, emulsion, foam, gel, lotion, milk, mousse, ointment, paste, powder, spray, or suspension. The cosmetic composition can be any colored cosmetic used on the skin, hair, eyes, or lips, such as concealing sticks, foundation, stage make-up, mascara (cake or cream), eye shadow (liquid, pomade, powder, stick, pressed or cream), hair color, lipsticks, lip gloss, kohl pencils, eye liners, blushers, eyebrow pencils, and cream powders. Other exemplary cosmetic compositions include, but are not limited to, nail enamel, skin glosser stick, hair sprays, face powder, leg-makeup, insect repellent lotion, nail enamel remover, perfume lotion, and shampoos of all types (gel or liquid). In addition, the claimed compositions can be used in shaving cream (concentrate for aerosol, brushless, lathering), hair groom, cologne stick, cologne, cologne emollient, bubble bath, body lotion (moisturizing, cleansing, analgesic, astringent), body wash, cleanser, soaps, body butter, creams, balms, serums, masks, after shave lotion, after bath milk, sunscreen lotion, and other personal care formulations.

The amount of the structural colorant that is present in the cosmetic or personal care formulations is dependent on type of cosmetic or personal care formulation, the intended color, and the intended use. More colorant can be used to create higher intensity, or provide higher coverage, or correction. In some embodiments, the cosmetic or personal care formulation contains greater than 0 wt % up to about 99.9 wt % of the structural colorant based upon the total weight of the formulation. This may include about 0.01 wt % to about 80 wt %; about 5 wt % to about 50 wt %; or about 5 wt % to about 20 wt %.

The balance of the formulation includes a carrier and optionally other additives such as preservatives, antioxidants, fragrances, other colorants, and fillers. For example, in addition to the structural colorants, the cosmetic or personal care formulations may include, carriers, excipients, emulsifiers, surfactants, preservatives, fragrances, perfume oils, thickeners, polymers, gel formers, dyes, absorption pigments, photoprotective agents, consistency regulators, antioxidants, antifoams, antistats, resins, solvents, solubility promoters, neutralizing agents, stabilizers, sterilizing agents, propellants, drying agents, opacifiers, cosmetically active ingredients, hair polymers, hair and skin conditioners, graft polymers, water-soluble or dispersible silicone-containing polymers, bleaches, care agents, colorants, tinting agents, tanning agents, humectants, refatting agents, collagen, protein hydrolyzates, lipids, emollients and softeners, tinting agents, tanning agents, bleaches, keratin-hardening substances, antimicrobial active ingredients, photofilter active ingredients, repellant active ingredients, hyperemic substances, keratolytic and keratoplastic substances, antidandruff active ingredients, antiphlogistics, keratinizing substances, active ingredients which act as antioxidants and/or as free-radical scavengers, skin moisturizing or humectants substances, refatting active ingredients, deodorizing active ingredients, sebostatic active ingredients, plant extracts, antierythematous or antiallergic active ingredients and mixtures thereof.

Illustrative additives for the cosmetic or personal care formulations, or by way of further explanation of the above materials, may also include, fatty alcohols, esters of fatty acids, natural or synthetic triglycerides including glyceryl esters and derivatives, waxes, pearlescent waxes, hydrocarbon oils, silicones or siloxanes, fluorinated or perfluorinated oils, emulsifiers, super-fatting agents, surfactants, consistency regulators or thickeners, rheology modifiers, polymers, cationic surfactants, deodorizing active ingredients, anti-dandruff agents, hydrotropic agents, preservatives, bacteria-inhibiting agents, fragrances, and other adjuvants.

### Fatty Alcohols

Illustrative fatty alcohols include, but are not limited to, guerbet alcohols based on fatty alcohols having from 6 to 18, preferably from 8 to 10 carbon atoms including cetyl alcohol, stearyl alcohol, cetearyl alcohol, oleyl alcohol, octyidodecanol, benzoate of C₁₂-C₁₅ alcohols, acetylated lanolin alcohol, and the like.

### Esters of Fatty Acids

Illustrative esters of fatty acids include, but are not limited to, esters of linear C₆-C₂₄ fatty acids with linear C₃-C₂₄ alcohols, esters of branched C₆-C₁₃-carboxylic acids with linear C₆-C₂₄ fatty alcohols, esters of linear C₆-C₂₄ fatty acids with branched alcohols, especially 2-ethylhexanol, esters of hydroxycarboxylic acids with linear or branched C₆-C₂₂ fatty alcohols, especially dioctyl malates, esters of linear and/or branched fatty acids with polyhydric alcohols (for example propylene glycol, dimer diol or trimer triol) and/or Guerbet alcohols, for example caproic acid, caprylic acid, 2-ethylhexanoic acid, capric acid, lauric acid, isotridecanoic acid, myristic acid, palmitic acid, palmitoleic acid, stearic acid, isostearic acid, oleic acid, elaidic acid, petroselinic acid, linoleic acid, linolenic acid, elaeostearic acid, arachidic acid, gadoleic acid, behenic acid and erucic acid and technical-grade mixtures thereof (obtained, for example, in the pressure removal of natural fats and oils, in the reduction of aldehydes from Roelen's oxosynthesis or in the dimerisation of unsaturated fatty acids) with alcohols, for example, isopropyl alcohol, caproic alcohol, capryl alcohol, 2-ethylhexyl alcohol, capric alcohol, lauryl alcohol, isotridecyl alcohol, myristyl alcohol, cetyl alcohol, palmoleyl alcohol, stearyl alcohol, isostearyl alcohol, oleyl alcohol, elaidyl alcohol, petroselinyl alcohol, linoyl alcohol, linolenyl alcohol, elaeostearyl alcohol, arachidyl alcohol, gadoleyl alcohol, behenyl alcohol, erucyl alcohol, bras sidyl alcohol, and mixtures of any tow or more thereof. The esters may be obtained, for example, in the high-pressure hydrogenation of technical-grade methyl esters based on fats and oils or aldehydes from Roelen's oxosynthesis and as monomer fractions in the dimerisation of unsaturated fatty alcohols. Examples of such ester oils are isopropylmyristate, isopropylpalmitate, isopropylstearate, isopropyl isostearate, isopropyloleate, n-butylstearate, n-hexyllaurate, n-decyloleate, iso-octylstearate, iso-nonylstearate, isononyl isononanoate, 2-ethylhexylpalmitate, 2-hexyllaurate, 2-hexyldecylstearate, 2-octyidodecylpalmitate, oleyloleate, oleylerucate, erucyloleate, erucylerucate, cetearyl octanoate, cetyl palmitate, cetyl stearate, cetyl oleate, cetyl behenate, cetyl acetate, myristyl myristate, myristyl behenate, myristyl oleate, myristyl stearate, myristyl palmitate, myristyl lactate, propylene glycol dicaprylate/caprate, stearyl heptanoate, diisostearyl malate, octyl hydroxystearate, or mixtures of any two or more thereof.

### Natural or Synthetic Di or Tri-Glycerides

Illustrative natural or synthetic triglycerides include, but are not limited to, di- or tri-glycerides, based on C₆-C₁₈ fatty acids, modified by reaction with other alcohols (caprylic/capric triglyceride, wheat germ glycerides, etc.). Fatty acid esters of polyglycerin (polyglyceryl-n such as polyglyceryl-4 caprate, polyglyceryl-2 isostearate, etc. or castor oil, hydrogenated vegetable oil, sweet almond oil, wheat germ oil, sesame oil, hydrogenated cottonseed oil, coconut oil, avocado oil, corn oil, hydrogenated castor oil, shea butter, cocoa butter, soybean oil, mink oil, sunflower oil, safflower oil, macadamia nut oil, olive oil, hydrogenated tallow, apricot kernel oil, hazelnut oil, and borago oil, or mixtures of any two or more thereof.

### Waxes

Illustrative waxes include, but are not limited to, esters of long-chain acids and alcohols as well as compounds having wax-like properties, e.g., carnauba wax, beeswax (white or yellow), lanolin wax, candellila wax, ozokerite, japan wax, paraffin wax, microcrystalline wax, ceresin, cetearyl esters wax, synthetic beeswax, or hydrophilic waxes such as cetearyl alcohol or partial glycerides, and mixtures of any two or more thereof.

### Pearlescent Waxes

Illustrative pearlescent waxes include, but are not limited to, alkylene glycol esters, especially ethylene glycol distearate; fatty acid alkanolamides, especially coco fatty acid diethanolamide; partial glycerides, especially stearic acid monoglyceride; esters of polyvalent, unsubstituted or hydroxy-substituted carboxylic acids with fatty alcohols having from 6 to 22 carbon atoms, especially long-chained esters of tartaric acid; fatty substances, for example fatty alcohols, fatty ketones, fatty aldehydes, fatty ethers and fatty carbonates, which in total have at least 24 carbon atoms, especially laurone and distearyl ether; fatty acids, such as stearic acid, hydroxystearic acid or behenic acid, ring-opening products of olefin epoxides having from 12 to 22 carbon atoms with fatty alcohols having from 12 to 22 carbon atoms and/or polyols having from 2 to 15 carbon atoms and from 2 to 10 hydroxy groups, and mixtures of any two or more thereof.

### Hydrocarbon Oils

Illustrative hydrocarbon oils include, but are not limited to, mineral oil (light or heavy), petrolatum (yellow or white), microcrystalline wax, paraffinic and isoparaffinic compounds, hydrogenated isoparaffinic molecules as polydecenes and polybutene, hydrogenated polyisobutene, squalane, isohexadecane, isododecane and others from plant and animal kingdom, or mixtures of any two or more thereof.

### Silicones or Siloxanes

Illustrative silicones and siloxanes include, but are not limited to, dimethylpolysiloxanes, methylphenylpolysiloxanes, cyclic silicones, and also amino-, fatty acid-, alcohol-, polyether-, epoxy-, fluorine-, glycoside- and/or alkyl-modified silicone compounds, which at room temperature may be in either liquid or resinous form. Linear polysiloxanes, dimethicone (Dow Corning 200 fluid, Rhodia Mirasil DM), dimethiconol, cyclic silicone fluids, cyclopentasiloxanes volatiles (Dow Corning 345 fluid), and phenyltrimethicone (Dow Corning 556 fluid). Also suitable are simethicones, which are mixtures of dimethicones having an average chain length of from 200 to 300 dimethylsiloxane units with hydrogenated silicates. A detailed survey by Todd et al. of suitable volatile silicones may in addition be found in Cosm. Toil. 91, 27 (1976). Mixtures of any two or more such materials are also contemplated.

### Fluorinated or Perfluorinated Oils

Illustrative fluorinated or perfluorinated oils include, but are not limited to, perfluorhexane, dimethylcyclohexane, ethylcyclopentane, polyperfluoromethyl isopropyl ether, and mixtures of any two or more thereof.

### Emulsifiers

Illustrative emulsifiers include, but are not limited to, carbocyclic acids and their salts: alkaline soap of sodium, potassium and ammonium, metallic soap of calcium or magnesium, organic basis soap such as Lauric, palmitic, stearic and oleic acid etc.; alkyl phosphates or phosphoric acid esters, acid phosphate, diethanolamine phosphate, potassium cetyl phosphate; ethoxylated carboxylic acids or polyethyleneglycol esters, and polyethylene glycol (“PEG”) acrylates; linear fatty alcohols having from 8 to 22 carbon atoms, branched from 2 to 30 mol of ethylene oxide and/or from 0 to 5 mol propylene oxide with fatty acids having from 12 to 22 carbon atoms and with alkylphenols having from 8 to 15 carbon atoms in the alkyl group; fatty alcohol polyglycolethers such as laureth-n, ceteareth-n, steareth-n, and oleth-n; fatty acid polyglycolethers such as PEG-n stearate, PEG-n oleate, and PEG-n cocoate; monoglycerides and polyol esters; C₁₂-C₂₂ fatty acid mono- and di-esters of addition products of from 1 to 30 mol of ethylene oxide with polyols; fatty acid and polyglycerol esters such as monostearate glycerol, diisostearoyl polyglyceryl-3-diisostearates, polyglyceryl-3-diisostearates, triglyceryl diisostearates, polyglyceryl-2-sesquiisostearates, or polyglyceryl dimerates; fatty acid polyglycolesters such as monostearate diethylene glycol, fatty acid and polyethylene glycol esters, fatty acid and saccharose esters such as sucro esters, glycerol and saccharose esters such as sucro glycerides; sorbitol and sorbitan, sorbitan mono- and di-esters of saturated and unsaturated fatty acids having from 6 to 22 carbon atoms and ethylene oxide addition products; polysorbate-n series, sorbitan esters such as sesquiisostearate, sorbitan, PEG-(6)-isostearate sorbitan, PEG-(10)-sorbitan laurate, PEG-17-dioleate sorbitan, glucose derivatives, C.sub.8-C.sub.22 alkyl-mono and oligoglycosides and ethoxylated analogues with glucose being preferred as the sugar component; oil-water emulsifiers such as methyl gluceth-20 sesquistearate, sorbitan stearate/sucrose cocoate, methyl glucose sesquistearate, and cetearyl alcohol/cetearyl glucoside; water oil emulsifiers such as methyl glucose dioleate/methyl glucose isostearate; sulfates and sulfonated derivatives, dialkylsulfosuccinates, dioctyl succinate, alkyl lauryl sulfonate, linear sulfonated parafins, sulfonated tetraproplyne sulfonate, sodium lauryl sulfates, amonium and ethanol-amine lauryl sulfates, lauyl ether sulfates, sodium laureth sulfates, sulfosuccinates, aceyl isothionates, alkanolamide sulfates, taurines, methyl taurines, imidazole sulfates; amine derivatives, amine salts, ethoxylated amines, oxide amine with chains containing an heterocycle such as alkyl imidazolines, pyridine derivatives, isoquinoteines, cetyl pyridinium chlorure, cetyl pyridinium bromide, quaternary ammonium such as cetyltrimethylbroide amonium broide (CTBA), stearylalkonium; amide derivatives, alkanolamides such as acylamide DEA, ethoxylated amides such as PEG-n acylamide, oxydeamide; polysiloxane/polyalkyl/polyether copolymers and derivatives, dimethicone, copolyols, silicone polyethylene oxide copolymer, silicone glycol copolymer; propoxylated or POE-n ethers (Meroxapols); polaxamers or poly-(oxyethylene)m-block-poly(oxypropylene)n-block(oxyethylene); zwitterionic surfactants that carry at least one quaternary ammonium group and at least one carboxylate and/or sulfonate group in the molecule; zwitterionic surfactants that are especially suitable are betaines, such as N-alkyl-N,N dimethylammonium glycinates, cocoalkyldimethylammonium glycinate, N-acylaminopropyl-N,N-dimethylammonium glycinates, cocoacylaminopropyldimethylammonium glycinate and 2 alkyl-3-carboxymethyl-3-hydroxyethylimidazolines each having from 8 to 18 carbon atoms in the alkyl or acyl group and also cocoacylaminoethylhydroxy-ethylcarboxymethylglycinate, N-alkylbetaine, N-alkylaminobetaines; alkylimidazolines, alkylopeptides, lipoaminoacides, self emulsifying bases; and mixtures of any two or more such compounds from the plurality of those substance classes and substances listed herein.

Illustrative non-ionic emulsifiers include, but are not limited to, PEG-6 beeswax (and) PEG-6 stearate (and) polyglyceryl-2-isostearate [Apifac], glyceryl stearate (and) PEG-100 stearate. [Arlacel 165], PEG-5 glyceryl stearate [arlatone 983 S], sorbitan oleate (and) polyglyceryl-3 ricinoleate. [Arlacel 1689], sorbitan stearate and sucrose cocoate [arlatone 2121], glyceryl stearate and laureth-23 [Cerasynth 945], cetearyl alcohol and ceteth-20 [Cetomacrogol Wax], cetearyl alcohol and colysorbate 60 and PEG-150 and stearate-20-[Polawax GP 200, Polawax NF], cetearyl alcohol and cetearyl polyglucoside [Emulgade PL 1618], cetearyl alcohol and ceteareth-20 [Emulgade 1000NI, Cosmowax], cetearyl alcohol and PEG-40 castor oil [Emulgade F Special], cetearyl alcohol and PEG-40 castor oil and sodium cetearyl sulfate [Emulgade F], stearyl alcohol and steareth-7 and steareth-10 [Emulgator E 2155], cetearyl alcohol and szeareth-7 and steareth-10 [Emulsifying wax U.S.N.F], glyceryl stearate and PEG-75 stearate [Gelot 64], propylene glycol ceteth-3 acetate. [Hetester PCS], propylene glycol isoceth-3 acetate [Hetester PHA], cetearyl alcohol and ceteth-12 and oleth-12 [Lanbritol Wax N 21], PEG-6 stearate and PEG-32 stearate [Tefose 1500], PEG-6 stearate and ceteth-20 and steareth-20 [Tefose 2000], PEG-6 stearate and ceteth-20 and glyceryl stearate and steareth-20 [Tefose 2561], glyceryl stearate and ceteareth-20 [Teginacid H, C, X].

Illustrative anionic emulsifiers include, but are not limited to, anionic emulsifiers such as PEG-2 stearate SE, glyceryl stearate SE [Monelgine, Cutina KD], propylene glycol stearate [Tegin P], cetearyl Alcohol and Sodium cetearyl sulfate [Lanette N. Cutina LE, Crodacol GP], cetearyl alcohol and sodium lauryl sulfate [Lanette W], trilaneth-4 phopshate and glycol stearate and PEG-2 stearate [Sedefos 75], glyceryl stearate and sodium lauryl Sulfate [Teginacid Special]. While illustrative cationic acid bases include, but are not limited to, cetearyl alcohol and cetrimonium bromide.

The emulsifiers may be used in an amount of, for example, from 1 to 30% by weight, especially from 4 to 20% by weight and preferably from 5 to 10% by weight, based on the total weight of the composition. When formulated in oil-water emulsions, the amount of such emulsifier system could represent 5% to 20% of the oil phase.

### Super-Fatting Agents

Illustrative super fatting agents include, but are not limited to, lanolin and lecithin and also polyethoxylated or acrylated lanolin and lecithin derivatives, polyol fatty acid esters, monoglycerides and fatty acid alkanolamides, the latter simultaneously acting as foam stabilisers.

### Surfactants

Illustrative surfactants, that is to say surfactants especially well tolerated by the skin, include, but are not limited to, include fatty alcohol polyglycol ether sulfates, monoglyceride sulfates, mono- and/or di-alkyl sulfosuccinates, fatty acid isethionates, fatty acid sarcosinates, fatty acid taurides, fatty acid glutamates, .alpha.-olefin sulfonates, ethercarboxylic acids, alkyl oligoglucosides, fatty acid glucamides, alkylamidobetaines and/or protein fatty acid condensation products, the latter preferably being based on wheat proteins.

### Consistency Regulators/Thickeners and Rheology Modifiers

Illustrative consistency regulators/thickeners and rheology modifiers include, but are not limited to, silicon dioxide, magnesium silicates, aluminium silicates, polysaccharides or derivatives thereof for example hyaluronic acid, xanthan gum, guar-guar, agar-agar, alginates, carraghenan, gellan, pectines, or modified cellulose such as hydroxycellulose, hydroxypropyl-methylcellulose. In addition polyacrylates or homopolymer of reticulated acrylic acids and polyacrylamides, carbomer (carbopol types 980, 981, 1382, ETD 2001, ETD2020, Ultrez 10) or Salcare range such as Salcare SC80 (steareth-10 alkyl ether/acrylates copolymer), Salcare SC81 (acrylates copolymer), Salcare SC91 and Salcare AST (sodium acrylates copolymer/PPG-1 trideceth-6), sepigel 305 (polyacrylamide/laureth-7), Simulgel NS and Simulgel EG (hydroxyethyl acrylate/sodium acryloyidimethyl taurate copolymer), Stabilen 30 (acrylates/vinyl isodecanoate crosspolymer), Pemulen TR-1 (acrylates/C10-30 alkyl acrylate crosspolymer), Luvigel EM (sodium acrylates copolymer), Aculyn 28 (acrylates/beheneth-25 methacrylate copolymer), and mixtures of any two or more thereof.

### Polymers

Illustrative cationic polymers include, but are not limited to, cationic cellulose derivatives, for example a quaternised hydroxymethyl cellulose obtainable under the name Polymer JR 400 from Amerchol, cationic starches, copolymers of diallylammonium salts and acrylamides, quaternised vinylpyrrolidone/vinyl imidazole polymers, for example Luviquata (BASF), condensation products of polyglycols and amines, quaternised collagen polypeptides, for example lauryidimonium hydroxypropyl hydrolyzed collagen (LamequataL/Grunau), quaternised wheat polypeptides, polyethyleneimine, cationic silicone polymers, for example amidomethicones, copolymers of adipic acid and dimethylaminohydroxypropyldiethylenetriamine (Cartaretin/Sandoz), copolymers of acrylic acid with dimethyldiallylammonium chloride (Merquat 550/Chemviron), polyaminopolyamides, as described, for example, in FR-A-2 252 840, and the crosslinked water-soluble polymers thereof, cationic chitin derivatives, for example of quaternised chitosan, optionally distributed as microcrystals; condensation products of dihaloalkyls, for example dibromobutane, with bisdialkylamines, for example bisdimethylamino-1,3-propane, cationic guar gum, for example Jaguar C-17, Jaguar C-16 from Celanese, quaternised ammonium salt polymers, for example Mirapol A-15, Mirapol AD-1, Mirapol AZ-1 from Miranol.

Illustrative anionic, zwitterionic, amphoteric, and non-ionic polymers include, but are not limited to, vinyl acetate/crotonic acid copolymers, vinyl-pyrrolidone/vinyl acrylate copolymers, vinyl acetate/butyl maleate/isobornyl acrylate copolymers, methyl vinyl ether/maleic anhydride copolymers and esters thereof, uncrosslinked polyacrylic acids and polyacrylic acids crosslinked with polyols, acrylamidopropyl-trimethylammonium chloride/acrylate copolymers, octyl acrylamide/methyl methacrylatetert. butylaminoethyl methacrylate/2-hydroxypropyl methacrylate copolymers, polyvinylpyrrolidone, vinylpyrrolidone/vinyl acetate copolymers, vinylpyrrolidone/dimethylaminoethyl methacrylate/vinyl caprolactam terpolymers and also optionally derivatised cellulose ethers and silicones.

### Cationic Surfactants

Illustrative cationic surfactants include, but are not limited to, cetyl trimethyl ammonium bromide (CTAB), dimethicone copolyols, amidomethicones, acrylamidopropyltrimonium chloride/Acrylamide copolymer, guar hydroxypropyl trimonium chloride, hydroxycetyl hydroxyethyl dimonium chloride quaternium compounds as listed in International Cosmetic Ingredient Dictionary and Handbook, 7.sup.th Edition 1997, for example Quaternium-80, polyquaternium compounds, as listed in International Cosmetic Ingredient Dictionary and Handbook, 7.sup.th Edition 1997, for example polyquaternium-5, polyquaternium-6, polyquaternium-7, polyquaternium-10, polyquaternium-11, polyquaternium-17, polyquaternium-18, polyquaternium-24 or polyquaternium-27, polyquaternium-28, polyquaternium-32, polyquaternium-37, and mixtures of any two or more thereof.

### Biogenic Active Ingredients

Illustrative bioactive agents include, but are not limited to, tocopherol, tocopherol acetate, tocopherol palmitate, ascorbic acid, deoxyribonucleic acid, retinol, bisabolol, allantoin, phytantriol, panthenol, AHA acids, amino acids, ceramides, pseudoceramides, essential oils, plant extracts and vitamin complexes, or mixtures of any two or more thereof.

### Deodorising Active Ingredients

Illustrative deodorising active ingredients include, but are not limited to, antiperspirants, for example aluminium chlorohydrates (see J. Soc. Cosm. Chem. 24, 281 (1973)), under the trade mark Locrona of Hoechst AG, Frankfurt (FRG), there is available commercially, for example, an aluminium chlorohydrate corresponding to formula Al₂(OH)₅Cl.2.5H₂O, the use of which is especially preferred (see J. Pharm. Pharmacol. 26, 531 (1975)). Besides the chlorohydrates, it is also possible to use aluminium hydroxyacetates and acidic aluminium/zirconium salts. Esterase inhibitors may be added as further deodorising active ingredients. Such inhibitors are preferably trialkyl citrates, such as trimethyl citrate, tripropyl citrate, triisopropyl citrate, tributyl citrate and especially triethyl citrate (Hydagen CAT, Henkel), which inhibit enzyme activity and hence reduce odour formation. Further substances that come into consideration as esterase inhibitors are sterol sulfates or phosphates, for example lanosterol, cholesterol, campesterol, stigmasterol and sitosterol sulfate or phosphate, dicarboxylic acids and esters thereof, for example glutaric acid, glutaric acid monoethyl ester, glutaric acid diethyl ester, adipic acid, adipic acid monoethyl ester, adipic acid diethyl ester, malonic acid and malonic acid diethyl ester and hydroxycarboxylic acids and esters thereof, for example citric acid, malic acid, tartaric acid or tartaric acid diethyl ester. Antibacterial active ingredients that influence the germ flora and kill or inhibit the growth of sweat-decomposing bacteria can likewise be present in the preparations (especially in stick preparations). Examples include chitosan, phenoxyethanol and chlorhexidine gluconate. 5-chloro-2-(2,4-dichlorophenoxy)-phenol (Triclosan, Irgasan, Ciba Specialty Chemicals Inc.) has also proved especially effective.

### Anti-Dandruff Agents

Illustrative anti-dandruff agents include, but are not limited to, climbazole, octopirox and zinc pyrithione. Customary film formers include, for example, chitosan, microcrystalline chitosan, quaternised chitosan, polyvinylpyrrolidone, vinylpyrrolidone/vinyl acetate copolymers, polymers of quaternary cellulose derivatives containing a high proportion of acrylic acid, collagen, hyaluronic acid and salts thereof and similar compounds.

### Hydrotropic Agents

Illustrative hydrotropic agents for the improvement of flow behaviour include, but are not limited to, ethoxylated or non ethoxylated mono-alcohols, diols or polyols with a low number of carbon atoms or their ethers (e.g. ethanol, isopropanol, 1,2-dipropanediol, propyleneglycol, glyerin, ethylene glycol, ethylene glycol monoethylether, ethylene glycol monobutylether, propylene glycol monomethylether, propylene glycol monoethylether, propylene glycol monobutylether, diethylene glycol monomethylether; diethylene glycol monoethylether, diethylene glycol monobutylether and similar products). The polyols for that purpose comprise preferably 2 to 15 carbon atoms and at least two hydroxy groups. The polyols may also contain further functional groups, especially amino groups, and/or may be modified with nitrogen. Typical examples are as follows: glycerol, alkylene glycols, for example ethylene glycol, diethylene glycol, propylene glycol, butylene glycol, hexylene glycol and also polyethylene glycols having an average molecular weight of from 100 to 1000 Dalton; technical oligoglycerol mixtures having an intrinsic degree of condensation of from 1.5 to 10, for example technical diglycerol mixtures having a diglycerol content of from 40 to 50% by weight; methylol compounds, such as, especially, trimethylolethane, trimethylolpropane, trimethylolbutane, pentaerythritol and dipentaerythritol; lower alkyl-glucosides, especially those having from 1 to 8 carbon atoms in the alkyl radical, for example methyl and butyl glucoside; sugar alcohols having from 5 to 12 carbon atoms, for example sorbitol or mannitol; sugars having from 5 to 12 carbon atoms, for example glucose or saccharose; amino sugars, for example glucamine; dialcohol amines, such as diethanolamine or 2-amino-1,3-propanediol.

### Preservatives

Illustrative preservatives include, but are not limited to, methyl-, ethyl-, propyl-, butyl-parabens, benzalkonium chloride, 2-bromo-2-nitro-propane-1,3-diol, dehydroacetic acid, diazolidinyl urea, 2-dichloro-benzyl alcohol, dmdm hydantoin, formaldehyde solution, methyldibromoglutanitrile, phenoxyethanol, sodium hydroxymethylglycinate, imidazolidinyl urea, triclosan and mixtures of any two or more thereof.

### Bacteria-Inhibiting Agents

Illustrative bacteria-inhibiting agents include, but are not limited to, include those that have a specific action against gram-positive bacteria, such as 2,4,4′-trichloro-2′-hydroxydiphenyl ether, chlorhexidine (1,6-di(4-chlorophenyl-biguanido)hexane) or TCC (3,4,4′-trichlorocarbanilide). A large number of aromatic substances and ethereal oils also have antimicrobial properties. Typical examples are the active ingredients eugenol, menthol and thymol in clove oil, mint oil and thyme oil. A natural deodorising agent of interest is the terpene alcohol farnesol (3,7,11-trimethyl-2,6,10-dodecatrien-1-ol), which is present in lime blossom oil. Glycerol monolaurate has also proved to be a bacteriostatic agent. The amount of the additional bacteria-inhibiting agents present is usually from 0.1 to 2% by weight, based on the solids content of the preparations.

### Perfume Oils

Illustrative perfume oil include, but are not limited to, mixtures of natural and/or synthetic aromatic substances. Illustrative natural aromatic substances are, for example, extracts from blossom (lilies, lavender, roses, jasmine, neroli, ylang-ylang), from stems and leaves (geranium, patchouli, petitgrain), from fruit (aniseed, coriander, carraway, juniper), from fruit peel (bergamot, lemons, oranges), from roots (mace, angelica, celery, cardamom, costus, iris, calmus), from wood (pinewood, sandalwood, guaiacum wood, cedarwood, rosewood), from herbs and grasses (tarragon, lemon grass, sage, thyme), from needles and twigs (spruce, pine, Scots pine, mountain pine), from resins and balsams (galbanum, elemi, benzoin, myrrh, olibanum, opoponax). Illustrative animal raw materials also come into consideration, for example civet and castoreum. Illustrative synthetic aromatic substances are, for example, products of the ester, ether, aldehyde, ketone, alcohol or hydrocarbon type. Illustrative aromatic substance compounds of the ester type are, for example, benzyl acetate, phenoxyethyl isobutyrate, p-tert-butylcyclohexyl acetate, linalyl acetate, dimethylbenzylcarbinyl acetate, phenylethyl acetate, linalyl benzoate, benzyl formate, ethylmethylphenyl glycinate, allylcyclohexyl propionate, styrallyl propionate and benzyl salicylate. Illustrative ethers include, for example, benzyl ethyl ether; the aldehydes include, for example, the linear alkanals having from 8 to 18 hydrocarbon atoms, citral, citronellal, citronellyl oxyacetaldehyde, cyclamen aldehyde, hydroxycitronellal, lilial and bourgeonal; the ketones include, for example, the ionones, isomethylionone and methyl cedryl ketone; the alcohols include, for example, anethol, citronellol, eugenol, isoeugenol, geraniol, linalool, phenyl ethyl alcohol and terpinol; and the hydrocarbons include mainly the terpenes and balsams. It is preferable, however, to use mixtures of various aromatic substances that together produce an attractive scent. Illustrative ethereal oils of relatively low volatility, which are chiefly used as aroma components, are also suitable as perfume oils, e.g. sage oil, camomile oil, clove oil, melissa oil, oil of cinnamon leaves, lime blossom oil, juniper berry oil, vetiver oil, olibanum oil, galbanum oil, labolanum oil and lavandin oil. Preference is given to the use of bergamot oil, dihydromyrcenol, lilial, lyral, citronellol, phenyl ethyl alcohol, hexyl cinnamaldehyde, geraniol, benzyl acetone, cyclamen aldehyde, linalool, boisambrene forte, ambroxan, indole, hedione, sandelice, lemon oil, tangerine oil, orange oil, allyl amyl glycolate, cyclovertal, lavandin oil, muscatel sage oil, damascone, bourbon geranium oil, cyclohexyl salicylate, vertofix coeur, iso-E-Super, Fixolide NP, evernyl, iraldein gamma, phenylacetic acid, geranyl acetate, benzyl acetate, rose oxide, romillat, irotyl and floramat alone or in admixture with one another.

### Other Adjuvants

Illustrative other adjuvants include, but are not limited to anti-foams, such as silicones, structurants, such as maleic acid, solubilisers, such as ethylene glycol, propylene glycol, glycerol or diethylene glycol, opacifiers, such as latex, styrene/PVP or styrene/acrylamide copolymers, propellants, such as propane/butane mixtures, N2O, dimethyl ether, CO2, N2 or air, so-called coupler and developer components as oxidation dye precursors, reducing agents, such as thioglycolic acid and derivatives thereof, thiolactic acid, cysteamine, thiomalic acid or mercaptoethanesulfonic acid, or oxidising agents, such as hydrogen peroxide, potassium bromate or sodium bromate. Illustrative insect repellents are, for example, N,N-diethyl-m-toluamide, 1,2-pentanediol or insect repellent 3535; suitable self-tanning agents are, for example, dihydroxyacetone and/or erythrulose or dihydroxy acetone and/or dihydroxy acetone precursors as described in WO 01/85124 and/or erythrulose.

### Structural Colorants

The structural colorants that are used in the cosmetics and person care products described herein are porous metal oxide spheres that are typically micron-scaled. The coloration is produced due to the structure of the material and not its chemical makeup. In the present technology, it has been found that cosmetics and personal care formulations/products may be prepared using certain porous metal oxide spheres which exhibit high quality color in bulk. The spheres provide color visible in the bulk.

In some embodiments, the structural colorants impart a viewing angle-independent color to the formulations, where the observed color of the cosmetic or personal care formulation does not changes as the viewing angle changes. In other words, the cosmetic or personal care formulation has a color that is independent of the orientation of the sphere. Such angle-independent color may be the result of disordered spheres.

In some embodiments the structural colorants impart a viewing angle-dependent color to the formulations, where depending on the angle at which the formulation is viewed, the color may be appear to change to be different from another angle. Such angle-dependent color may be the result of ordered regions of pores on the individual porous metal oxide microspheres. That is, the entire particle, or at least a region thereof, may have an ordered, repeating array of pores compared to a disorder arrange of pores on the sphere where no repeating pattern exists over even a partial region of the microsphere.

The angle-dependent character imparted by the porous metal oxide spheres to the cosmetic or personal care formulation may provide color travel (i.e. change in color) as the viewing angle changes. The porous metal oxide microspheres described herein may impart two-quadrant, three-quadrant, or four-quadrant color change in Lab color space (including parameters such as L*, a*, b*, C, and h°). In other words, the observed color of a cosmetic or personal care formulation as described herein may appear to change from one quadrant to the next.

In some embodiments, the porous metal oxide spheres impart a white color to the cosmetic or person care formulation without the use of titanium dioxide as a whitener and opacifier.

In some embodiments, the porous metal oxide spheres in combination with other additives to the formulation allow for manipulation of the color shade of the composition without adding other colorants or a different porous metal oxide sphere to the formulation. For example, the addition of a high refractive index cosmetic ingredient relative to the base formulation may change the shade of the formulation from yellow to orange.

Also provided herein are nail finishes/polishes. The nail finishes also include a carrier and porous metal oxide microspheres, where the carrier includes a curable polymer. The polymer may be cured by solvent evaporation or by ultraviolet light. Illustrative polymers may include, but are not limited to, polyacrylates, polymethacrylates, polyurethanes, polyesters, celluloses, polyphthalates, and blends of two or more thereof. The nail finishes may also include one or more of fillers; solvents such as alcohols, acetates, phthalates, and aromatics; or other pigments such as iron oxides, ferric ferrocyanide, titanium dioxide, Red No. 7, Red. No. 6, Red No. 33, or Yellow No. 5.

According to any of the embodiments herein, the porous metal oxide spheres may have, e.g., an average diameter of from about 0.5 μm to about 100 μm and an average porosity of from about 0.10 to about 0.90 or from about 0.10 to about 0.80. The porous metal oxide spheres may also have more than one population of pores each having, e.g., an average pore diameter, wherein each population has a different average pore diameter; and wherein the average pore diameters are, e.g., from about 50 nm to about 999 nm. For example, the porous metal oxide spheres may have a first population of pores having an average pore diameter of from about 50 nm to about 999 nm and a second population of pores having average pore diameter of from about 50 nm to about 999 nm, wherein the first and second average pore diameters are different. According to any of the embodiments herein, the porous metal oxide spheres may have an average diameter of about 1 μm to about 75 μm. This may include about 2 μm to about 70 μm; about 3 μm to about 65 μm; about 4 μm to about 60 μm; about 5 μm to about 55 μm; or about 5 μm to about 50 μm. This may further include from any of about 5 μm, about 6 μm, about 7 μm, about 8 μm, about 9 μm, about 10 μm, about 11 μm, about 12 μm, about 13 μm, about 14 μm, or about 15 μm to any of about 16 μm, about 17 μm, about 18 μm, about 19 μm, about 20 μm, about 21 μm, about 22 μm, about 23 μm, about 24 μm, or about 25 μm.

According to any of the embodiments herein, the porous metal oxide spheres may have an average porosity of any of about 0.10, about 0.12, about 0.14, about 0.16, about 0.18, about 0.20, about 0.22, about 0.24, about 0.26, about 0.28, about 0.30, about 0.32, about 0.34, about 0.36, about 0.38, about 0.40, about 0.42, about 0.44, about 0.46, about 0.48 about 0.50, about 0.52, about 0.54, about 0.56, about 0.58, or about 0.60 to any of about 0.62, about 0.64, about 0.66, about 0.68, about 0.70, about 0.72, about 0.74, about 0.76, about 0.78, about 0.80, or about 0.90.

According to any of the embodiments herein, the porous metal oxide spheres may have an average pore diameter of from any of about 50 nm, about 60 nm, about 70 nm, about 80 nm, about 100 nm, about 120 nm, about 140 nm, about 160 nm, about 180 nm, about 200 nm, about 220 nm, about 240 nm, about 260 nm, about 280 nm, about 300 nm, about 320 nm, about 340 nm, about 360 nm, about 380 nm, about 400 nm, about 420 nm, or about 440 nm to any of about 460 nm, about 480 nm, about 500 nm, about 520 nm, about 540 nm, about 560 nm, about 580 nm, about 600 nm, about 620 nm, about 640 nm, about 660 nm, about 680 nm, about 700 nm, about 720 nm, about 740 nm, about 760 nm, about 780 nm, or about 800 nm.

According to any of the embodiments herein, the metal oxide of the porous metal oxide spheres may be silica, titania, alumina, zirconia, ceria, iron oxides, zinc oxide, indium oxide, tin oxide, chromium oxide, or a mixture of any two or more thereof. According to any of the embodiments herein, the metal oxide of the porous metal oxide microspheres may be silica, titania, alumina, or a mixture of any two or more thereof. According to a preferred embodiment, the metal oxide of the porous metal oxide microspheres may be silica, titania, alumina, or a mixture of any two or more thereof. According to a more preferred embodiment, the metal oxide of the porous metal oxide microspheres may be silica, titania or a mixture of any two or more thereof.

To describe the structural colorant further, details of their preparation are provided below. The structural colorants that are used in the cosmetic and personal care formulations are prepared using a polymeric sacrificial template. In the process of making the structural colorants, an aqueous colloid dispersion containing polymer particles and a metal oxide is prepared, the polymer particles typically being nano-scaled. The aqueous colloidal dispersion is mixed with a continuous oil phase, for instance within a microfluidic device, to produce a water-in-oil emulsion. Emulsion aqueous droplets are prepared, collected and dried to form microspheres containing polymer nanoparticles and metal oxide. The polymer particles (e.g., nanospheres) are then removed, for instance via calcination, to provide spherical, metal oxide particles (e.g., microspheres) containing a high degree of porosity pores that are typically nanoscaled. The spheres may contain uniform pore diameters, a result of the polymer particles being spherical and monodisperse.

In some embodiments, droplet formation and collection occurs within a microfluidic device. Microfluidic devices are for instance narrow channel devices having a micron-scaled droplet junction adapted to produce uniform size droplets connected to a collection reservoir. Microfluidic devices for example contain a droplet junction having a channel width of from about 10 μm to about 100 μm. The devices are for instance made of polydimethylsiloxane (PDMS) and may be prepared for example via soft lithography. An emulsion may be prepared within the device via pumping an aqueous dispersed phase and oil continuous phase at specified rates to the device where mixing occurs to provide emulsion droplets. Alternatively, an oil-in-water emulsion may be employed.

In some embodiments, vibrating nozzle techniques may be employed. In these techniques, a liquid dispersion is prepared; droplets are formed and are dropped into a bath of a continuous phase. The droplets are then dried, followed by removal of the polymer. Vibrating nozzle equipment is available from Büchi and comprises for instance a syringe pump and a pulsation unit. Vibrating nozzle equipment may also comprise a pressure regulation valve.

The polymer particles for instance have an average diameter of from about 50 nm to about 999 nm, and they are monodisperse.

Suitable template polymers include thermoplastic polymers. For example, template polymers are selected from the group consisting of poly(meth)acrylic acid, poly(meth)acrylates, polystyrenes, polyacrylamides, polyvinyl alcohol, polyvinyl acetate, polyesters, polyurethanes, polyethylene, polypropylene, polylactic acid, polyacrylonitrile, polyvinyl ethers, derivatives thereof, salts thereof, copolymers thereof and combinations thereof. For example, the polymer is selected from the group consisting of polymethyl methacrylate, polyethyl methacrylate, poly(n-butyl methacrylate), polystyrene, poly(chloro-styrene), poly(alpha-methylstyrene), poly(N-methylolacrylamide), styrene/methyl methacrylate copolymer, polyalkylated acrylate, polyhydroxyl acrylate, polyamino acrylate, polycyanoacrylate, polyfluorinated acrylate, poly(N-methylolacrylamide), polyacrylic acid, polymethacrylic acid, methyl methacrylate/ethyl acrylate/acrylic acid copolymer, styrene/methyl methacrylate/acrylic acid copolymer, polyvinyl acetate, polyvinylpyrrolidone, polyvinylcaprolactone, polyvinylcaprolactam, derivatives thereof, salts thereof, and combinations thereof.

In certain embodiments, polymer templates include polystyrenes, including polystyrene and polystyrene copolymers. Polystyrene copolymers include copolymers with water-soluble monomers, for example polystyrene/acrylic acid, polystyrene/poly(ethylene glycol) methacrylate, and polystyrene/styrene sulfonate.

Present metal oxides include oxides of transition metals, metalloids and rare earths, for example silica, titania, alumina, zirconia, ceria, iron oxides, zinc oxide, indium oxide, tin oxide, chromium oxide, mixed metal oxides, combinations thereof, and the like.

The wt/wt (weight/weight) ratio of polymer particles to metal oxide is for instance from about 0.1/1 to about 10.0/1 or from about 0.5/1 to about 10.0/1.

The continuous oil phase comprises for example an organic solvent, a silicone oil or a fluorinated oil. According to the invention “oil” means an organic phase immiscible with water. Organic solvents include hydrocarbons, for example, heptane, hexane, toluene, xylene, and the like, as well as alkanols such as methanol, ethanol, propanol, etc.

The emulsion droplets are collected, dried and the polymer is removed. Drying is performed for instance via microwave irradiation, in a thermal oven, under vacuum, in the presence of a desiccant, or a combination thereof.

Polymer removal may be performed for example via calcination, pyrolysis or with a solvent (solvent removal). Calcination is performed in some embodiments at temperatures of at least about 200° C., at least about 500° C., at least about 1000° C., from about 200° C. to about 1200° C. or from about 200° C. to about 700° C. The calcining can be for a suitable period, e.g., from about 0.1 hour to about 12 hours or from about 1 hour to about 8.0 hours. In other embodiments, the calcining can be for at least about 0.1 hour, at least about 1 hour, at least about 5 hours or at least about 10 hours.

Alternatively, a liquid dispersion comprising polymer particles and a metal oxide is formed with an oil dispersed phase and a continuous water phase to form an oil-in-water emulsion. The oil droplets may be collected and dried as are aqueous droplets.

Alternatively, a liquid dispersion of polymer particles and a metal oxide is prepared and is spray-dried to form the polymer template spheres without forming a liquid-in-liquid emulsion. In certain embodiments of spray-drying techniques, a liquid solution or dispersion is fed (e.g. pumped) to an atomizing nozzle associated with a compressed gas inlet. The feed is pumped through the atomizing nozzle to form liquid droplets. The droplets are surrounded by a pre-heated gas in an evaporation chamber, resulting in evaporation of solvent to produce solid particles. The dried particles are carried by the drying gas through a cyclone and deposited in a collection chamber. Gases include nitrogen and/or air. In an embodiment of a present spray-drying process, a liquid feed contains a water or oil phase, polymer particles and optionally metal oxide. Provided are polymer spheres containing polymer nanospheres with optionally metal oxide in the interstitial spaces between the polymer nanospheres. The polymer spheres define the interstitial spaces. Spray-drying techniques include ink jet spray-drying methods and equipment.

In present spray-drying techniques, air may be considered a continuous phase with a dispersed liquid phase (a liquid-in-gas emulsion). In certain embodiments, spray-drying comprises an inlet temperature of from any of about 100° C., about 105° C., about 110° C., about 115° C., about 120° C., about 130° C., about 140° C., about 150° C., about 160° C. or about 170° C. to any of about 180° C., about 190° C., about 200° C., about 210° C., about 215° C. or about 220° C. In some embodiments a pump rate (feed flow rate) of from any of about 1 mL/min, about 2 mL/min, about 5 mL/min, about 6 mL/min, about 8 mL/min, about 10 mL/min, about 12 mL/min, about 14 mL/min or about 16 mL/min to any of about 18 mL/min, about 20 mL/min, about 22 mL/min, about 24 mL/min, about 26 mL/min, about 28 mL/min or about 30 mL/min is employed. Spray-drying techniques are disclosed for example in US2016/0170091.

In certain embodiments of spray-drying techniques, a feed solution or dispersion is fed to an atomizing nozzle associated with a compressed gas inlet. The feed is pumped through the atomizing nozzle to form liquid droplets. The droplets are surrounded by a pre-heated gas in an evaporation chamber, resulting in evaporation of solvent to produce solid particles. The dried particles are carried by the drying gas through a cyclone and deposited in a collection chamber. Gases include nitrogen and/or air. In a present spray-drying process, a liquid feed contains water, polymer particles and metal oxide.

The spheres are spherical or spherical-like and are typically micron-scaled, for example having average diameters from about 0.5 microns (μm) to about 100 μm. The polymer particles employed as a template are also spherical and are typically nano-scaled, having average diameters for instance from about 50 nm to about 999 nm. The metal oxide employed may also be in particle form, which particles may be nano-scaled.

The metal oxide of the dispersion may be provided as metal oxide or may be provided from a metal oxide precursor, for instance via a sol-gel technique.

Drying of the polymer/metal oxide droplets and removal of the polymer provides spheres having voids (pores). In general, in the present processes, each droplet provides a single sphere. The pore diameters are dependent on the size of the polymer particles. Some “shrinkage” or compaction may occur upon removal of the polymer, providing pore sizes somewhat smaller than the original polymer particle size, for example from about 10% to about 40% smaller than the polymer particle size. The pore diameters vary as the polymer particle size varies (is polydisperse).

The polymer sphere diameters of the polymer spheres and the pore diameters of the porous spheres may be for example bimodal, trimodal, quadrimodal, etc. Pore diameters may range in some embodiments from about 50 nm to about 999 nm.

The average porosity of the present metal oxide spheres may be relatively high, for example from about 0.10 or about 0.30 to about 0.80 or about 0.90. Average porosity of a sphere means the total pore volume, as a fraction of the volume of the entire sphere. Average porosity may be called “volume fraction.”

In some embodiments, a porous sphere may have a solid core (center) where the porosity is in general towards the exterior surface of the sphere. In other embodiments, a porous sphere may have a hollow core where a major portion of the porosity is towards the interior of the sphere. In other embodiments, the porosity may be distributed throughout the volume of the sphere. In other embodiments, the porosity may exist as a gradient, with higher porosity towards the exterior surface of the sphere and lower or no porosity (solid) towards the center; or with lower porosity towards the exterior surface and with higher or complete porosity (hollow) towards the center.

For any porous sphere, the average sphere diameter is larger than the average pore diameter, for example, the average microsphere diameter is at least about 25 times, at least about 30 times, at least about 35 times, or at least about 40 times larger than the average pore diameter.

In some embodiments, the ratio of average sphere diameter to average pore diameter is for instance from any of about 40/1, about 50/1, about 60/1, about 70/1, about 80/1, about 90/1, about 100/1, about 110/1, about 120/1, about 130/1, about 140/1, about 150/1, about 160/1, about 170/1, about 180/1 or about 190/1 to any of about 200/1, about 210/1, about 220/1, about 230/1, about 240/1, about 250/1, about 260/1, about 270/1, about 280/1, about 290/1, about 300/1, about 310/1, about 320/1, about 330/1, about 340/1 or about 350/1.

Polymer template spheres comprising polydisperse polymer spheres may provide, when the polymer is removed, metal oxide spheres having pores that in general have varied pore diameters.

Without wishing to be bound by theory, it is believed that bulk samples of spheres exhibit saturated color with reduced unwanted light scattering when porosity and/or microsphere diameter and/or pore diameter are within a certain range. Color properties of a bulk sample are important, as colorants are employed in bulk, for instance in a paint, an ink, a coating, a cosmetic or a material for a medical application or a security application. In some embodiments, white spheres are desirable, for example for use as white colorants.

The porous spheres comprise mainly metal oxide, that is, they may consist essentially of or consist of metal oxide. Advantageously, a bulk sample of the porous spheres exhibits color observable by the human eye. A light absorber may also be present in the spheres, which may provide a more saturated observable color. Absorbers include inorganic and organic pigments, for example a broadband absorber such as carbon black. Absorbers may for instance be added by physically mixing the spheres and the absorbers together or by including the absorbers in the droplets to be dried. For carbon black, controlled calcination may be employed to produce carbon black in situ from polymer decomposition. A present sphere may exhibit no observable color without added light absorber and exhibit observable color with added light absorber.

The porous spheres may be employed as colorants for example for aqueous formulations, oil-based formulations, inks, coatings formulations, foods, plastics, cosmetics formulations or materials for medical applications or security applications. Coatings formulations include for instance automotive coatings, architectural coatings, varnishes, and the like.

The present porous metal oxide spheres may exhibit angle-dependent color or angle-independent color. “Angle-dependent” color means that observed color has dependence on the angle of incident light on a sample or on the angle between the observer and the sample. “Angle-independent” color means that observed color has substantially no dependence on the angle of incident light on a sample or on the angle between the observer and the sample.

Angle-independent color may be achieved for example with the use of polydisperse polymer spheres. Angle-independent color may also be achieved when a step of drying the liquid droplets to provide polymer template spheres is performed quickly, not allowing the polymer spheres to become ordered. Angle-dependent color may be achieved when a step of drying the liquid droplets is performed slowly.

For instance, the porous spheres may comprise from about 60.0 wt % (weight percent) to about 99.9 wt % metal oxide and from about 0.1 wt % to about 40.0 wt % of one or more light absorbers, based on the total weight of the spheres.

Removal of a monodisperse population of polymer spheres provides porous metal oxide spheres having a corresponding population of pores having an average pore diameter. Removal of more than one monodisperse population of polymer spheres (polydisperse polymer nanospheres) provides porous metal oxide spheres having corresponding populations of pores having different average pore diameters. That is, porous metal oxide spheres having more than one population of pores, each having an average pore diameter, wherein each population has a different average pore diameter and wherein the average pore diameters are from about 50 nm to about 999 nm.

The present invention, thus generally described, will be understood more readily by reference to the following examples, which are provided by way of illustration and are not intended to be limiting of the present invention.

### EXAMPLES

The following examples are prophetic and are formulations illustrative of the scope of the present technology. As used in the examples, q.s. indicates that the component is likely to be used in an amount sufficient for the type of component, but exact amounts are not provided. The porous metal oxide microspheres of the formulation of the invention have been prepared according to US 2019/0076809 A1 and US 2019/0076810 A1, which are incorporated herein by reference.

### Example 1. Sunscreen Formulation

| Part | Component | wt % |
|---|---|---|
| A | DI Water(q.s. to 100%) | 43.60 |
| | Panthenol | 1.00 |
| | PEG-8 | 2.50 |
| | Disodium EDTA | 0.10 |
| | Polyurethane-39 | 2.50 |
| B | Octyltriazone | 2.00 |
| | Bemotrizinol | 3.00 |
| | Propylheptyl Caprylate | 5.00 |
| | Dicaprylyl Carbonate | 5.00 |
| C | Ceteareth-25 | 2.50 |
| | Polyglyceryl-3 Distearate | 2.50 |
| | PEG-7 Hydrogenated Castor Oil | 0.50 |
| | Stearyl Alcohol | 2.50 |
| | Dimethicone | 1.50 |
| | Hydrogenated Polyisobutene | 1.50 |
| | Tocopherol Acetate | 0.50 |
| | Bisabolol | 1.00 |
| D | DI Water | 10.00 |
| | Bisoctrizole | 10.00 |
| E | Porous Titania Microspheres | 2.60 |
| F | Fragrance (Orange Nectar AD78-00997) | 0.20 |
| | Preservatives | q.s. |

Parts A, B, and C are individually mixed and heated to 75-80° C. Part B is then added to Part C under agitation. The B/C mixture is then added to Part A under homogenizing conditions for 2-3 minutes and low speed and cooled to 50° C. Part D is separately mixed and added to the B/C/A mixture. Then Part is added and the entire mixture homogenized until uniform. Finally, Part F is added in its constituent part and the entire formulation is mixed.

### Example 2. Cream Foundation

| Part | INCI Name | wt % |
|---|---|---|
| A | DI Water | 60.60 |
| | Methylpropanediol | 5.00 |
| | Magnesium Aluminum Silicate | 0.60 |
| | Xanthan Gum | 0.40 |
| B | Cetearyl Olivate (and) Sorbitan Olivate | 4.00 |
| | Hydrogenated Olive Oil (and) Olea Europaea | 2.00 |
| | (Olive) Fruit Oil (and) Olea Europaea (Olive) | |
| | Oil Unsaponifiables | |
| | Caprylic/Capric Triglyceride (and) Di-PPG-3 | 7.00 |
| | Myristyl Ether Adipate (and) Sorbitan | |
| | Isostearate | |
| | Meadowfoam Estolide (and) Meadowfoam Delta | 2.00 |
| | Lactone | |
| | Isodecyl Neopentanoate | 5.00 |
| | Antioxidants | q.s. |
| | Preservatives | q.s. |
| C | Kaolin | 0.50 |
| | Polymethyl Methacrylate | 4.00 |
| | Mica (and) Lauroyl Lysine | 3.00 |
| | Porous Titania Microspheres | 5.90 |

The deionized water (DI Water) and methylpropandiol are added to a main vessel and blended, followed by slowly adding the magnesium aluminum silicate and xanthan gum. In a separate vessel the Part B components are mixed and heated to 60-70° C. until the mixture is uniform, at which point Part B is added under homogenization to Part A. After pulverization of the Part C components in blending equipment, Part C is added to the Part A/B mixture until a uniform color is obtained, at which point the temperature is lowered to 40° C.

### Example 3. Eyeliner Formulation

| Part | INCI Name | wt % |
|---|---|---|
| A | DI Water | (q.s to 100%) |
| | VP/Methacrylamide/Vinyl Imidazole Copolymer | 20.00 |
| | Panthenol | 1.00 |
| | Glycerin (and) Glyceryl Acrylate/Acrylic Acid | 10.00 |
| | Co-polymer (and) Propylene Glycol (and) | |
| | PVM/MA Copolymer | |
| | Antioxidants | q.s. |
| | Preservatives | q.s. |
| B | Xanthan Gum | 1.50 |
| C | Porous Titania Microspheres | 11.25 |

In a suitable container all Part A materials are combined and Part B is added under rapid agitation. After combination of A and B is complete, Part C is added to the mixture.

### Example 4. Crème Lipstick

| Part | INCI Name | wt % |
|---|---|---|
| A | Crambe Abyssinica Seed Oil | 17.86 |
| | Euphorbia Cerifera (Candelilla) Wax | 3.00 |
| | Copernicia Cerifera (Carnauba) Wax | 1.50 |
| | Beeswax | 1.00 |
| | Ceresine | 6.00 |
| | Microcrystalline Wax | 1.50 |
| | Oleyl Alcohol | 3.00 |
| | Isosteryl Palmitate | 4.25 |
| | Caprylic/Capric Triglyceride | 8.25 |
| | Bis-Diglyceryl Polyacyladipate-2 | 2.00 |
| | Acetylated Lanolin Alcohol | 2.50 |
| | Sorbitan Tristearate | 1.75 |
| | Ozokerite | 6.75 |
| | Glyceryl Monolaurate | 1.00 |
| | Antioxidants | q.s. |
| | Preservatives | q.s. |
| | UV Absorbers | q.s. |
| B | Meadowfoam Estolide | 2.00 |
| | Pentaerythrityl Tetraisostearate | 6.00 |
| C | Crambe Abyssinica Seed Oil | 20.00 |
| | Red 21* | 1.14 |
| | Porous Titania Microspheres | 10.50 |
| D | Fragrance | q.s. |

All Part A materials are added to a vessel and heated to about 85° C. with stirring until the mixture is melted and uniform. Part B is then pre-dispersed into Part A at about 82° C. for 30 minutes with gentle agitation, and while maintaining the temperature, Part C is added with gentle agitation. The overall mixture is then cooled to 75° C. and Part D is added.

### Example 5. Pressed Powder Eyeshadow

| Part | INCI Name | wt % |
|---|---|---|
| A | Mica (and) Bismuth Oxychloride | 31.50 |
| | Kaolin | 15.00 |
| | Microcrystalline Cellulose | 12.50 |
| | Porous Titania Microspheres | 31.00 |
| B | Crambe Abyssinica Seed Oil (and) | |
| | Butyrospermum | |
| | Parkii (Shea Butter) Extract | 7.50 |
| | Simmondsia Chinensis (Jojoba) Seed Oil | 1.50 |
| | Cocos Nucifera (Coconut) Oil | 1.00 |
| | Antioxidant | q.s. |
| | Preservatives | q.s. |

Part A is thoroughly dry blended and dispersed. In a separate vessel Part B is pre-dispersed. Pre-dispersed Part B is then sprayed into Part A, the mixture is then pulverized and pressed.

### Example 6. Shampoo

| Part | INCI Name | wt % |
|---|---|---|
| A | DI Water | 15.40 |
| | Acrylates/Aminoacrylates/C10-30 Alkyl PEG- | 7.00 |
| | 20 Itaconate Copolymer | |
| B | DI Water | 15.00 |
| | Cocamide MEA | 0.50 |
| C | Sodium Laureth Sulfate | 35.70 |
| | Cocamidopropyl Betaine | 13.50 |
| | Disodium Laureth Sulfosuccinate | 7.80 |
| | Polyquaternium-87 | 1.84 |
| | Preservatives | q.s. |
| | Fragrance (Spring Flower # 0794029) | 0.50 |
| | Blue 1 (1% Aqueous Solution) | 0.25 |
| | Porous Titania Microspheres | 0.16 |
| D | Citric Acid (10% Aqueous Solution) | 2.35 |

Weigh out components of Phase A and Phase B separately and stir until the solution is homogenous. Add Phase B to Phase A and stir until uniform. Add Phase C to Phase AB and stir until uniform. Adjust pH to 5.6 with citric acid with constant stirring.

### Example 7. Loose Face Powder

| Part | INCI Name | wt % |
|---|---|---|
| A | Porous Titania Microspheres | 95 |
| | Magnesium Stearate | 1 |
| B | Shorea Stenoptera Seed Butter | 1.5 |
| | Coco-caprylate | 1 |
| | Caprylic/Capric Triglyceride | 0.5 |
| | Preservatives | q.s. |

Part A is thoroughly dry blended and dispersed. In a separate vessel, Part B is heated to 80° C. under agitation until uniform, and then Part B is sprayed into Part A and pulverized.

Parts A and B are separately stirred until the respective mixtures are homogenous. Part B is then added to Part A with stirring until uniform, at which time Part C is then added. The pH is adjusted to 5.6 by the addition of citric acid with stirring.

### Example 8. Nail Polish

| Part | INCI Name | wt % |
|---|---|---|
| A | Butyl Acetate (and) Ethyl Acetate (and) | 97.0 |
| | Nitrocellulose (and) Isopropyl Alcohol | |
| B | D&C Black 2 (10% dispersion) | 1.0 |
| | Porous Titania Microspheres | 5.00 |

Part A is charged to a vessel fitted with a mechanical mixer, and Part B is added slowly until the overall mixture is uniform.

A nail polish formulation containing 5 wt % titania microspheres with 0.1 wt % D&C Black 2 in nail enamel base. This formula exhibited a green color.

A 75 μm wet film of the nail polish exhibited a color with the values of L*=44.9, a*=−2.3, b*=−3.8, C*=4.4, and h°=239.2 at an illuminant angle of 45° and a measurement angle of 15°, quantified with a multi-angle spectrophotometer on the dry film. The measurement of the film was made over the black portion of a standard contrast card/opacity chart.

The titania microspheres used in the nail polish have a microsphere diameter range of 1-7 μm and an average pore size of 175 nm.

### Example 9. Lip Gloss

| Part | INCI Name | wt % |
|---|---|---|
| A | Mineral Oil (and) Ethylene/Propylene/Styrene | 84 |
| | Copolymer (and) Butylene/Ethylene/Styrene | |
| | Copolymer | |
| | Octyldodecanol | 6 |
| | Coco-Caprylate | 2 |
| | Cocoglycerides | 2 |
| B | Iron Oxides (Black, 10% dispersion in DISM) | 5.0 |
| C | Porous Titania Microspheres | 1.0 |
| D | Preservatives | q.s. |

In a suitable container all Part A materials are combined and Part B is added with mixing. After combination of A and B is complete, Part C is then added to the mixture.

A lip gloss containing 1 wt % titania microspheres and 0.5 wt % black iron oxide. This formula exhibited a green color.

A 75 μm wet film of the lip gloss exhibited a color with the values of L*=23.2, a*=−3.0, b*=−5.7, C*=6.4, and h°=242.3 at an illuminant angle of 45° and a measurement angle of 15°, quantified with a multi-angle spectrophotometer on the wet film. The measurement of the film was made on the black portion of a standard contrast card/opacity chart.

The microspheres have a microsphere diameter range of 1-7 μm and an average pore size of 175 nm.

### Example 10. Lip Gloss

| Part | INCI Name | wt % |
|---|---|---|
| A | Mineral Oil (and) Ethylene/Propylene/Styrene | 89.8 |
| | Copolymer (and) Butylene/Ethylene/Styrene | |
| | Copolymer | |
| | Octyldodecanol | 6 |
| | Coco-Caprylate | 2 |
| | Cocoglycerides | 2 |
| C | Porous Silica Microspheres | 0.20 |
| D | Preservatives | q.s. |

In a suitable container all Part A materials are combined and Part B is added with mixing. After combination of A and B is complete, Part C is then added to the mixture.

A lip gloss containing 0.2 wt % silica microspheres exhibited no visible color/appeared transparent.

The microspheres used in the lip gloss formulation have a microsphere diameter range of 1-15 μm and an average pore size of 285 nm.

### Example 11. Shower Gel

| Part | INCI Name | wt % |
|---|---|---|
| A | Water | 64.57 |
| | Disodium EDTA | 0.10 |
| | Sodium Laureth Sulfate | 27.50 |
| | Cocamidopropyl Betaine | 1.00 |
| | Panthenol | 0.03 |
| B | Acrylates Copolymer | 6.00 |
| | Aminomethylpropanol | 0.60 |
| C | Porous Silica Microspheres | 0.20 |
| D | Preservatives | q.s. |

Weigh out components of Phase A and stir until the solution is homogenous. Add Acrylates Copolymer to Phase A and stir until uniform. Neutralize with Aminomethylpropanol. Add Phase C to Phase AB and stir until uniform.

A shower gel formulation containing 0.2 wt % silica microspheres exhibited a red color.

The microspheres used in the shower gel have a microsphere diameter range of 1-15 μm and an average pore size of 285 nm.

While certain embodiments have been illustrated and described, it should be understood that changes and modifications can be made therein in accordance with ordinary skill in the art without departing from the technology in its broader aspects as defined in the following claims.

The embodiments, illustratively described herein may suitably be practiced in the absence of any element or elements, limitation or limitations, not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the claimed technology. Additionally, the phrase “consisting essentially of” will be understood to include those elements specifically recited and those additional elements that do not materially affect the basic and novel characteristics of the claimed technology. The phrase “consisting of” excludes any element not specified.

The present disclosure is not to be limited in terms of the particular embodiments described in this application. Many modifications and variations can be made without departing from its spirit and scope, as will be apparent to those skilled in the art. Functionally equivalent methods and compositions within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled. It is to be understood that this disclosure is not limited to particular methods, reagents, compounds compositions or biological systems, which can of course vary. It is also to be understood that the terminology used herein is for the purpose of describing particular embodiments only, and is not intended to be limiting.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member.

All publications, patent applications, issued patents, and other documents referred to in this specification are herein incorporated by reference as if each individual publication, patent application, issued patent, or other document was specifically and individually indicated to be incorporated by reference in its entirety. Definitions that are contained in text incorporated by reference are excluded to the extent that they contradict definitions in this disclosure.

Summarizing the above findings, the present invention includes the following embodiments, wherein these include the specific combinations of embodiments as indicated by the respective interdependencies defined therein.

Embodiment (1): A cosmetic or personal care formulation comprising a carrier and porous metal oxide spheres wherein, the porous metal oxide spheres have an average diameter of about 0.5 μm to about 100 μm and an average porosity of about 0.10 to about 0.80; the porous metal oxide spheres have more than one population of pores each having an average pore diameter, wherein each population has a different average pore diameter and wherein the average pore diameters are from about 50 nm to about 999 nm.
Embodiment (2): The cosmetic or personal care formulation of embodiment (1), wherein the carrier comprises one or more of water, polymers, triglycerides, esters, light absorbers, pigments other than porous metal oxide spheres, waxes, alcohols, acids, bases, antioxidants, fragrances, preservatives, and vitamins.
Embodiment (3): The cosmetic or personal care formulation of any one of embodiments (1)-(2), wherein the carrier comprises one or more of polymers, triglycerides, esters, light absorbers, pigments other than porous metal oxide spheres, waxes, alcohols, acids, bases, antioxidants, fragrances, preservatives, and vitamins.
Embodiment (4): The cosmetic or personal care formulation of any one of embodiments (1)-(3) which is a cosmetic selected from lipstick, lip liner, lip gloss, eyeshadow, eyeliner, nail enamel, nail polish, concealer, fournation, or cream.
Embodiment (5): The cosmetic or personal care formulation of any one of embodiments (1)-(4) which is a personal care formulation selected from shampoo, body wash, cleanser, soap, body butter, lotion, cream, balm, serum, mask, or sunscreen.
Embodiment (6): The cosmetic or personal care formulation of any one of embodiments (1)-(5), wherein the porous metal oxide spheres are present in the formulation from greater than 0 wt % to about 99.9 wt %.
Embodiment (7): The cosmetic or personal care formulation of any one of embodiments (1)-(6), wherein the porous metal oxide spheres are present in the formulation at 0.01 wt % to about 80 wt %.
Embodiment (8): The cosmetic or personal care formulation of any one of embodiments (1)-(7), wherein the porous metal oxide spheres are present in the formulation at about 5 wt % to about 50 wt %.
Embodiment (9): The cosmetic or personal care formulation of any one of embodiments (1)-(8), wherein the porous metal oxide spheres are present in the formulation at about 5 wt % to about 25 wt %.
Embodiment (10): The cosmetic or personal care formulation of any one of embodiments (1)-(7) which is a nail enamel, wherein the carrier comprises a nail enamel base.
Embodiment (11): A cosmetic or personal care formulation comprising a carrier and porous metal oxide spheres having an ordered structure wherein, the formulation exhibits an angle-dependent color.
Embodiment (12): The cosmetic or personal care formulation of embodiment (11), wherein the porous metal oxide spheres have an average diameter of about 0.5 μm to about 100 μm and an average porosity of about 0.10 to about 0.80; the porous metal oxide spheres have more than one population of pores each having an average pore diameter, wherein each population has a different average pore diameter and wherein the average pore diameters are from about 50 nm to about 999 nm.
Embodiment (13): The cosmetic or personal care formulation of embodiment (11) or (12), wherein the ordered structure is a repeating pattern of pores over at least one region of each of the porous metal oxide spheres.
Embodiment (14): The cosmetic or personal care formulation of any one of embodiments (11)-(13), wherein the carrier comprises one or more of water, polymers, triglycerides, esters, light absorbers, pigments other than porous metal oxide spheres, waxes, alcohols, acids, bases, antioxidants, fragrances, preservatives, and vitamins.
Embodiment (15): The cosmetic or personal care formulation of any one of embodiments (11)-(13), wherein the carrier comprises one or more of polymers, triglycerides, esters, light absorbers, pigments other than porous metal oxide spheres, waxes, alcohols, acids, bases, antioxidants, fragrances, preservatives, and vitamins.
Embodiment (16): The cosmetic or personal care formulation of any one of embodiments (11)-(15) which is a cosmetic selected from lipstick, lip liner, lip gloss, eyeshadow, eyeliner, nail enamel, nail polish, concealer, fournation, or cream.
Embodiment (17): The cosmetic or personal care formulation of any one of embodiments (11)-(14) which is a personal care formulation selected from shampoo, body wash, cleanser, soap, body butter, lotion, cream, balm, serum, mask, or sunscreen.
Embodiment (18): The cosmetic or personal care formulation of any one of embodiments (11)-(17), wherein the porous metal oxide spheres are present in the formulation from greater than 0 wt % to about 99.9 wt %.
Embodiment (19): The cosmetic or personal care formulation of any one of embodiments (11)-(18), wherein the porous metal oxide spheres are present in the formulation at 0.01 wt % to about 80 wt %.
Embodiment (20): The cosmetic or personal care formulation of any one of embodiments (11)-(19), wherein the porous metal oxide spheres are present in the formulation at about 5 wt % to about 50 wt %.
Embodiment (21): The cosmetic or personal care formulation of any one of embodiments (11)-(20), wherein the porous metal oxide spheres are present in the formulation at about 5 wt % to about 25 wt %.
Embodiment (22): A formulation comprising a carrier and porous metal oxide spheres, wherein the formulation is a cosmetic or personal care formulation that is stimuli-responsive.
Embodiment (23): The formulation of embodiment (22), wherein the porous metal oxide spheres have an average diameter of about 0.5 μm to about 100 μm and an average porosity of about 0.10 to about 0.80; the porous metal oxide spheres have more than one population of pores each having an average pore diameter, wherein each population has a different average pore diameter and wherein the average pore diameters are from about 50 nm to about 999 nm.
Embodiment (24): The formulation of embodiment (22) or (23), wherein the formulation changes color in response to the stimuli selected from pH, pressure, ultraviolet light, visible, near-infra red light, temperature, or electric current.

1. A cosmetic or personal care formulation comprising a carrier and porous metal oxide spheres wherein, the porous metal oxide spheres have an average diameter of about 0.5 pm to about 100 pm and an average porosity of about 0.10 to about 0.80; the porous metal oxide spheres have more than one population of pores each having an average pore diameter, wherein each population has a different average pore diameter and wherein the average pore diameters are from about 50 nm to about 999 nm.

2. The cosmetic or personal care formulation of claim 1, wherein the carrier comprises one or more of water, polymers, triglycerides, esters, light absorbers, pigments other than porous metal oxide spheres, waxes, alcohols, acids, bases, antioxidants, fragrances, preservatives, and vitamins.

3. The cosmetic or personal care formulation of claim 1, wherein the carrier comprises one or more of polymers, triglycerides, esters, light absorbers, pigments other than porous metal oxide spheres, waxes, alcohols, acids, bases, antioxidants, fragrances, preservatives, and vitamins.

4. The cosmetic or personal care formulation of claim 1, which is a cosmetic selected from lipstick, lip liner, lip gloss, eyeshadow, eyeliner, nail enamel, nail polish, concealer, foundation, or cream.

5. The cosmetic or personal care formulation of claim 1, which is a personal care formulation selected from shampoo, body wash, cleanser, soap, body butter, lotion, cream, balm, serum, mask, or sunscreen.

6. The cosmetic or personal care formulation of claim 1, wherein the porous metal oxide spheres are present in the formulation from greater than 0 wt % to about 99.9 wt %.

7. The cosmetic or personal care formulation of claim 1, wherein the porous metal oxide spheres are present in the formulation at 0.01 wt % to about 80 wt %.

8. The cosmetic or personal care formulation of claim 1, wherein the porous metal oxide spheres are present in the formulation at about 5 wt % to about 50 wt %.

9. The cosmetic or personal care formulation of claim 1, wherein the porous metal oxide spheres are present in the formulation at about 5 wt % to about 25 wt %.

10. The cosmetic or personal care formulation of claim 1 which is a nail enamel, wherein the carrier comprises a nail enamel base.

11. A cosmetic or personal care formulation comprising a carrier and porous metal oxide spheres having an ordered structure wherein, the formulation exhibits an angle-dependent color.

12. The cosmetic or personal care formulation of claim 11, wherein the porous metal oxide spheres have an average diameter of about 0.5 pm to about 100 pm and an average porosity of about 0.10 to about 0.80; the porous metal oxide spheres have more than one population of pores each having an average pore diameter, wherein each population has a different average pore diameter and wherein the average pore diameters are from about 50 nm to about 999 nm.

13. The cosmetic or personal care formulation of claim 11, wherein the ordered structure is a repeating pattern of pores over at least one region of each of the porous metal oxide spheres.

14. The cosmetic or personal care formulation of claim 11, wherein the carrier comprises one or more of water, polymers, triglycerides, esters, light absorbers, pigments other than porous metal oxide spheres, waxes, alcohols, acids, bases, antioxidants, fragrances, preservatives, and vitamins.

15. The cosmetic or personal care formulation of claim 11, wherein the carrier comprises one or more of polymers, triglycerides, esters, light absorbers, pigments other than porous metal oxide spheres, waxes, alcohols, acids, bases, antioxidants, fragrances, preservatives, and vitamins.

16. The cosmetic or personal care formulation of claim 11 which is a cosmetic selected from lipstick, lip liner, lip gloss, eyeshadow, eyeliner, nail enamel, nail polish, concealer, foundation, or cream.

17. The cosmetic or personal care formulation of claim 11 which is a personal care formulation selected from shampoo, body wash, cleanser, soap, body butter, lotion, cream, balm, serum, mask, or sunscreen.

18. The cosmetic or personal care formulation of claim 11, wherein the porous metal oxide spheres are present in the formulation from greater than 0 wt % to about 99.9 wt %.

19. The cosmetic or personal care formulation of claim 11, wherein the porous metal oxide spheres are present in the formulation at 0.01 wt % to about 80 wt %.

20. The cosmetic or personal care formulation of claim 11, wherein the porous metal oxide spheres are present in the formulation at about 5 wt % to about 50 wt %.

21. The cosmetic or personal care formulation of claim 11, wherein the porous metal oxide spheres are present in the formulation at about 5 wt % to about 25 wt %.

22. A formulation comprising a carrier and porous metal oxide spheres, wherein the formulation is a cosmetic or personal care formulation that is stimuli-responsive.

23. The formulation of claim 22, wherein the porous metal oxide spheres have an average diameter of about 0.5 pm to about 100 pm and an average porosity of about 0.10 to about 0.80; the porous metal oxide spheres have more than one population of pores each having an average pore diameter, wherein each population has a different average pore diameter and wherein the average pore diameters are from about 50 nm to about 999 nm.

24. The formulation of claim 22, wherein the formulation changes color in response to the stimuli selected from pH, pressure, ultraviolet light, visible, near-infra red light, temperature, or electric current.
